(19) United States
(12) Patent Application Publication
(10) Pub. No.: US 2023/0347136 A1
(43) Pub. Date: Nov. 2, 2023

(54) SYSTEMS AND METHODS FOR CHRONIC LOWER BACK PAIN TREATMENT

(71) Applicant: Cymedica Orthopedics, Inc., Scottsdale, AZ (US)

(72) Inventors: Jessica Paparella-Wachtel, Scottsdale, AZ (US); Kereshmeh Shahriari, Scottsdale, AZ (US); Ryan Fig, Scottsdale, AZ (US); Erin Hart, Scottsdale, AZ (US); Dharmavirsinh Jhala, Scottsdale, AZ (US); Struan Coleman, Scottsdale, AZ (US)

(21) Appl. No.: 18/128,629

(22) Filed: Mar. 30, 2023

Related U.S. Application Data
(60) Provisional application No. 63/325,470, filed on Mar. 30, 2022.

(51) Int. Cl. A61N 1/04; A61N 1/36

(52) U.S. Cl. CPC A61N 1/0484; A61N 1/0452; A61N 1/0456; A61N 1/0476; A61N 1/36021; A61N 1/36034

(57) ABSTRACT

In some embodiments, the system includes a wearable garment that is able to couple to electrode panels to secure the electrode panels in a position to contract transversus abdominis muscles and lumbar multifidus muscles. In some embodiments, the system includes a controller that couples to at least one electrode panel to deliver electrical power to electrodes. In some embodiments, the controller executes program instructions that can independently contract transversus abdominis muscles and lumbar multifidus muscles or can contract the transversus abdominis muscles and lumbar multifidus muscles simultaneously. In some embodiments, the controller executes an NMES and a TENS program simultaneously. In some embodiments, the NMES and TENS are executed by the same electrode.

### CROSS-REFERENCE TO RELATED APPLICATION

This application claims the benefit and priority of U.S. Provisional Pat. Application No. 63/325,470, filed Mar. 30, 2022, which is incorporated herein by reference in its entirety.

### BACKGROUND

What is commonly referred to as lower back pain includes pain, muscle tension, or stiffness localized in the lumbar region and above the inferior gluteal folds, with or without sciatica. Lower back pain is defined as chronic when it persists for 12 weeks or more. About 20 percent of people affected by acute lower back pain develop chronic lower back pain with persistent symptoms at one year after initial symptoms. FIG. 1 illustrates the area of the back affected by chronic low back pain (“CLBP”).

CLBP is most diagnosed in the 40-69 age group and is the number one cause of work-related disability in people under 45, as well as the most common reason for people missing work. Lower back pain is the fifth most common reason for physician visits, and the treatment market for CLBP was valued at $6.29 billion in 2018. FIG. 2 shows an illustration of causes, risk factors, diagnosis, and current treatments for CLBP. In 2017, CLBP affected over 34 million people (~ 6% of the adult population) in the United States alone. However, only about 5 million people (~ 15%) of those affected are eligible for surgery.

CLBP is mainly due to mechanical instability in the spine caused by problems with one or more of neural control, passive elements (e.g., vertebral position, spinal locator, and spinal motion), and/or active elements (e.g., a spinal muscle activation pattern). The spinal structure is designed to maintain upright posture, absorb shock, and accommodate bipedal gait through three normal curves: cervical lordosis, thoracic kyphosis, and lumbar lordosis. Spinal alignment also depends on stabilizing structures such as the facet joints, spinal ligaments, and the intervertebral discs, as well as the muscles that provide dynamic stability by absorbing loading of the spine during activities. The core muscles, which include the transversus abdominis (TrA), the deepest abdominal muscle, provide critical dynamic stabilization for the lumbar spine. Posteriorly, the erector spinae muscles span multiple levels of the spine and provide for erect posture. The deepest compartment posteriorly consists of the lumbar multifidus (LM) muscles, which provide segmental stability to individual vertebrae and increase the stiffness of the spine during function. These deep compartment muscles are most important to provide essential stability to the spine.

Factors associated with CLBP include compromised dynamic stability and segmental stability of the lumbar spine and altered muscle activation to compensate for the reduced stability. Patients with CLBP do not sufficiently activate the deep lumbar stabilizing muscles of the TrA and LM which are essential for lumbar spinal stability. Muscle atrophy changes affecting the TrA and LM and motor control disturbances result in dynamic and segmental instability of the lumbar spine. This in turn may cause or perpetuate CLBP.

Atrophy changes impacting LM and TrA muscle groups result in segmental and dynamic instability of the lumbar spine and development of CLBP. Atrophied multifidus is seen in about 80% of CLBP patients. Unstable spine can result in lower back pain, LM and TrA muscles neural activation deficit, overloading of the joint, and re-injury leading to a continuing cycle of chronic lower back pain.

Electrical stimulation to elicit LM contraction via implanted wire electrodes has previously been found to be effective in decreasing back pain intensity and improving functional outcome scores in patients with LBP. However, there is a need in the art for a non-invasive solution to alleviate CLBP as opposed to resorting to more invasive procedures.

### SUMMARY

In some embodiments, the disclosure is directed to a system for alleviating chronic lower back pain. In some embodiments, the system comprises a wearable garment and/or a plurality of electrodes. In some embodiments, the plurality of electrodes are positioned on the wearable garment such that a neuromuscular electrical stimulation (NMES) pulse to each of the plurality of electrodes will contract transversus abdominis (TrA) muscles and lumbar multifidus (LM) muscles simultaneously. In some embodiments, the wearable garment is configured to enable one or more of the plurality of electrodes to be repositioned.

In some embodiments, the system further comprises a panel. In some embodiments, the panel comprises at least one of the plurality of electrodes. In some embodiments, the panel is configured to electrically couple to at least one of the plurality of electrodes. In some embodiments, the panel is configured to add rigidity to at least one of the plurality of electrodes. In some embodiments, the wearable garment is configured to secure the panel in position such that at least one of the plurality of electrodes contracts at least one of the TrA muscles and the LM muscles when an NMES pulse is applied to at least one of the plurality of electrodes.

In some embodiments, the system comprises a plurality of panels. In some embodiments, each of the plurality of panels comprise electrical contacts configured to physically couple to at least one of the plurality of electrodes. In some embodiments, each of the plurality of panels are configured to electrically couple to at least one of the plurality of electrodes. In some embodiments, the wearable garment is configured to secure each of the plurality of panels in a position such that an NMES pulse contracts the TrA muscles and/or the LM muscles.

In some embodiments, the plurality of panels includes one or more electrode panels. In some embodiments, the electrode panel comprises electrical connections and/or mechanical connections for two or more electrodes. In some embodiments, the mechanical connections are configured to position the two or more electrodes such that a neuromuscular electrical stimulation (NMES) pulse to each of the two or more electrodes will contract transversus abdominis (TrA) muscles. In some embodiments, the mechanical connections are configured to position the two or more electrodes such that a neuromuscular electrical stimulation (NMES) pulse to each of the two or more electrodes will contract lumbar multifidus (LM) muscles.

In some embodiments, a controller is configured to execute one or more program steps that include: execute, by one or more processors, a TrA pulse; stop, by the one or more processors, the TrA pulse; execute, by the one or more processors, a LM pulse; stop, by the one or more processors, the LM pulse; and execute, by the one or more processors, the TrA pulse and the LM pulse simultaneously.

In some embodiments, the electrode panel comprises rigidity properties and flexibility properties. In some embodiments, the rigidity properties enable the electrode panel to maintain a substantially same shape under an influence of gravity when rotated to various positions. In some embodiments, and the flexibility properties enable the electrode panel to bend an amount sufficient to ensure each of the two or more electrodes contact skin to contract transversus abdominis (TrA) muscles and/or lumbar multifidus (LM) muscles when the two or more electrodes are connected to the electrode panel. In some embodiments, the electrode panel comprises an electrical circuit configured to deliver electrical pulses to each of the two or more electrodes. In some embodiments, the electrode panel comprises a dock configured to enable a controller to be connected to a docking board. In some embodiments, the system comprises one or more of a controller and a garment. In some embodiments, the controller is configured to electrically couple to the electrode panel. In some embodiments, the garment comprises one or more mechanical couplings configured to removably secure the controller and/or the electrode panel in a position.

In some embodiments, the system for alleviating chronic lower back pain comprises a plurality of electrodes and/or a controller. In some embodiments, the controller is configured to execute a neuromuscular electrical stimulation (NMES) program configured to generate one or more NMES pulses to each of the plurality of electrodes. In some embodiments, the one or more NMES pulses comprise a transversus abdominis (TrA) pulse configured to contract TrA muscles. In some embodiments, the one or more NMES pulses comprise a lumbar multifidus (LM) pulse configured to contract LM muscles.

In some embodiments, the controller is configured to one or more program steps that include: execute, by one or more processors, the TrA pulse; stop, by the one or more processors, the TrA pulse; execute, by the one or more processors, the LM pulse; and stop, by the one or more processors, the LM pulse. In some embodiments, the controller is configured to execute transcutaneous electrical stimulation (TENS) and NMES programs. In some embodiments, wherein the controller is configured to execute transcutaneous electrical stimulation (TENS) and NMES programs to a same electrode simultaneously.

In some embodiments, the system includes a garment and/or one or more panels. In some embodiments, the controller is configured to electrically couple to at least one of the one or more panels. In some embodiments, the garment comprises one or more mechanical couplings configured to removably secure the one or more panels in one or more positions. In some embodiments, the one or more positions are configured to deliver the TrA pulse and/or the LM pulse. In some embodiments, the one or more panels comprise an electrical circuit configured to deliver electrical pulses to each of the plurality of electrodes. In some embodiments, the one or more panels comprises a dock configured to enable the controller to be connected to a docking board to execute one or more program steps.

### DRAWINGS DESCRIPTION

FIG. 1 illustrates the area of the back affected by chronic low back pain (CLBP).

FIG. 2 shows an illustration of causes, risk factors, diagnosis, and current treatments for CLBP.

FIG. 3 illustrates the role that the system plays in helping to alleviate pain associated with CLBP and return to function according to some embodiments.

FIGS. 4-8 show system electrode placement for CLPB treatment on a patient according to some embodiments.

FIGS. 9 and 10 show a wearable garment that includes a unique wrap, band, and/or belt design for placement on the user as shown in FIGS. 4-8 according to some embodiments of the system.

FIG. 11 illustrates an electronics system overview according to some embodiments.

FIG. 12 shows an electrode wiring diagram according to some embodiments.

FIG. 13 depicts rendered views of the assembled electrode panel as well as a thermoformed panel configuration according to some embodiments.

FIG. 14 shows options for connecting the docking board to the flexible circuit as well as a rendering of the flexible circuit material according to some embodiments.

FIG. 15 shows NMES waveform parameters utilized in ActivCore Therapy therapeutic protocol for a CLBP device described herein according to some embodiments.

FIGS. 16A and 16B show NMES/TENS combination waveform parameters according to some embodiments.

FIG. 17 depicts additional NMES/TENS combination waveform parameters according to some embodiments.

FIG. 18 depicts a change in transverse abdominus muscle thickness from rest to stimulation according to some embodiments.

FIG. 19 shows a change in lumbar multifidus muscle thickness from rest to stimulation according to some embodiments.

FIG. 20 illustrates a computer system enabling or comprising the systems and methods described herein in accordance with some embodiments.

FIG. 21 shows a flowchart of how the system implements a personalized dose of NMES therapy by measuring patient’s muscle strength, disease stage, and/or by using a patient database analytics and/or machine learning.

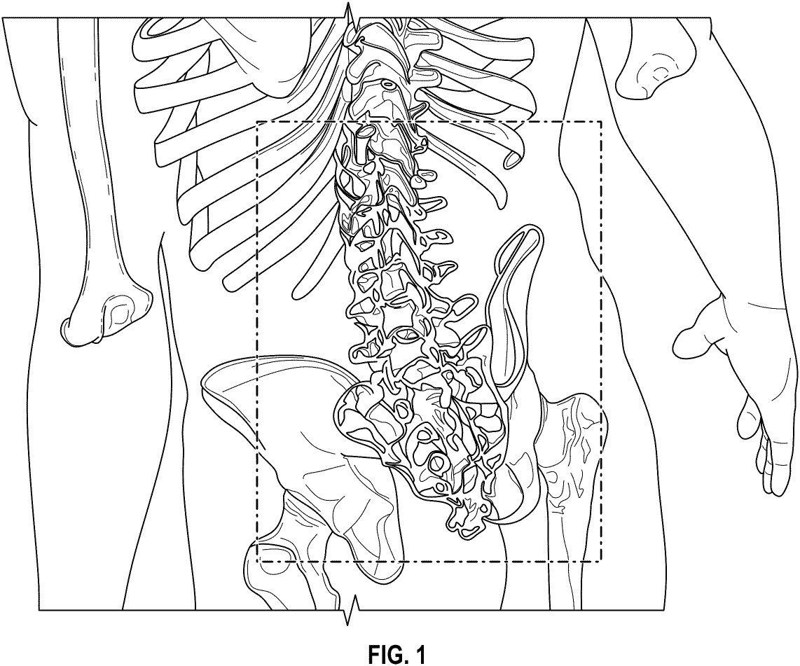

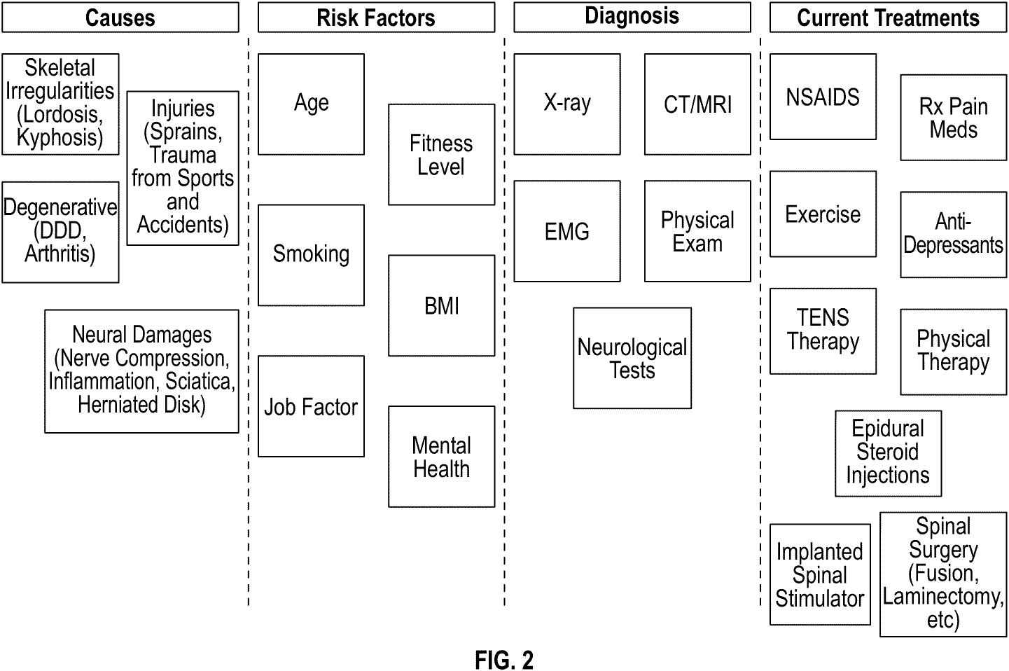

### DETAILED DESCRIPTION

In some embodiments, the system includes a non-invasive neuromuscular electrical stimulation (NMES) apparatus which includes surface electrodes configured to provide pain relief and therapeutic effects without surgical intervention. In some embodiments, the system is configured to provide lumbopelvic stabilizing rehabilitation to reinforce muscle strength and prevent muscle atrophy. In some embodiments, the system is configured to increase the strength of the deep lumbar stabilizing muscles. Muscle activation implemented by the system has empirically resulted in clinical improvements in patients with CLBP.

LM and TrA are the deepest muscles of the abdominal and back areas that may be difficult to activate from the surface of the skin. In some embodiments, the system is configured to transmit one or more of high intensity waveforms and wide electrical stimulation pulses to stimulate and activate one or more of these muscles. In some embodiments, the system comprises a controller configured to implement one or more program steps described herein. In some embodiments, a program step includes a step to implement a co-contraction and/or simultaneous stimulation of the LM and TrA muscles.

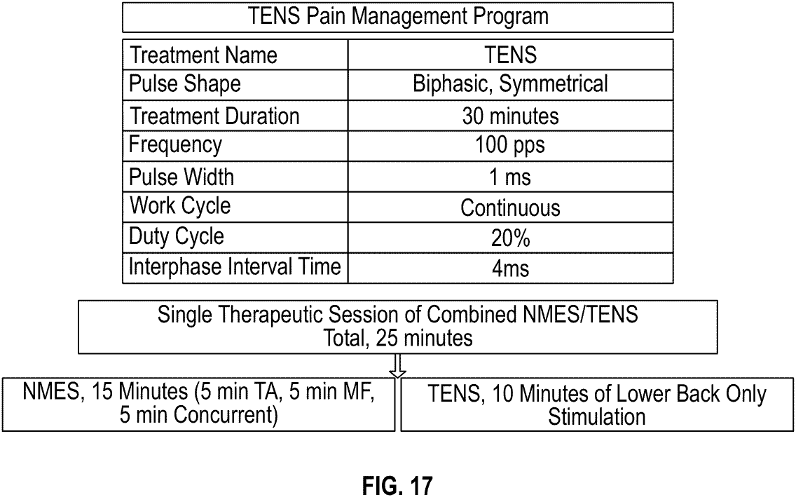

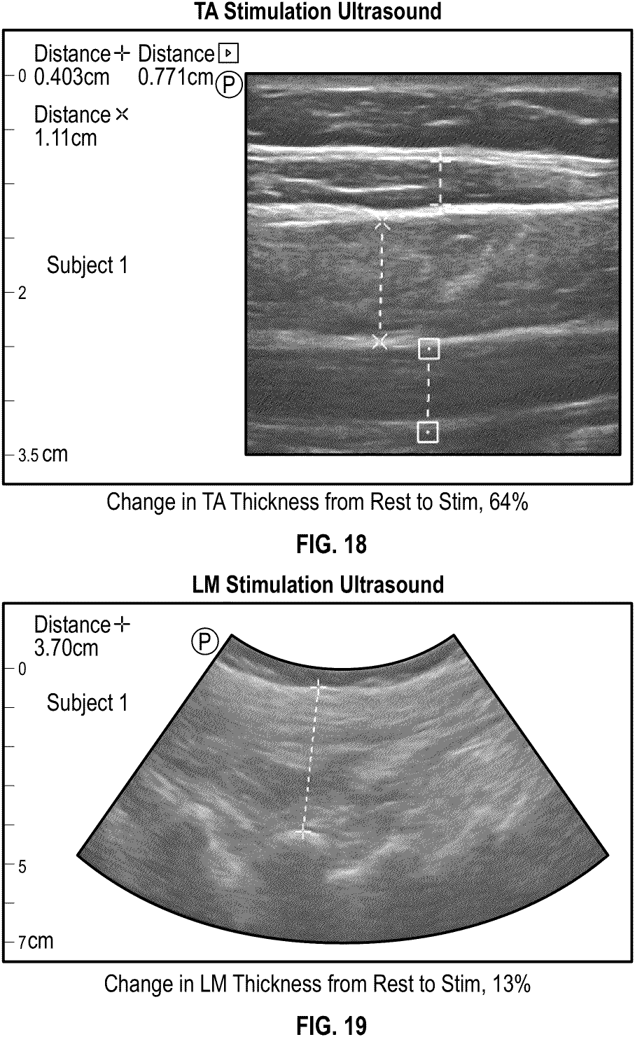

In some embodiments, the system (controller) is configured to implement a unique NMES waveform using skin contacting electrodes as a noninvasive intervention. In some embodiments, the system is configured to target the deep muscles of TrA and LM to overcome the difficulties associated with voluntary activation of these deep spinal stabilizing muscle groups. FIGS. 18 and 19 illustrate activation of the TrA and LM muscles according to some embodiments. In some embodiments, the controller is configured to implement a unique NMES waveform which includes a high intensity, monophasic waveform with a long pulse duration, and a unique polarization shape.

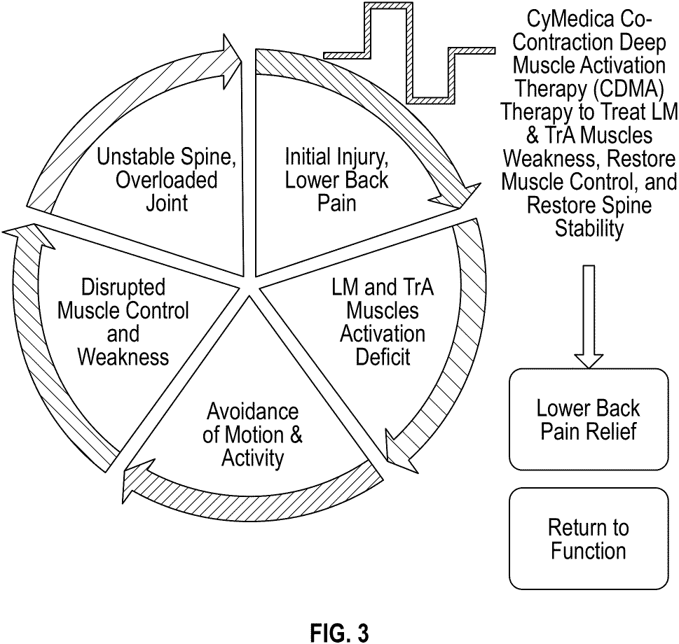

In some embodiments, the controller is configured to implement a co-contraction program configured to cause stimulation of the TrA and LM muscles independently, followed by stimulation of the TrA and LM muscles simultaneously, to provide a co-contraction effect of the core and lumbar spine. In some embodiments, the stimulation of TrA and LM muscles over time may strengthen muscles, stabilize the spinal structure, and/or facilitate effective pain relief associated with the CLBP. In some embodiments, the co-contraction program is configured to provide essential dynamic and segmental stability to the spine. FIG. 3 illustrates the role co-contraction program protocol plays in helping to alleviate CLBP according to some embodiments.

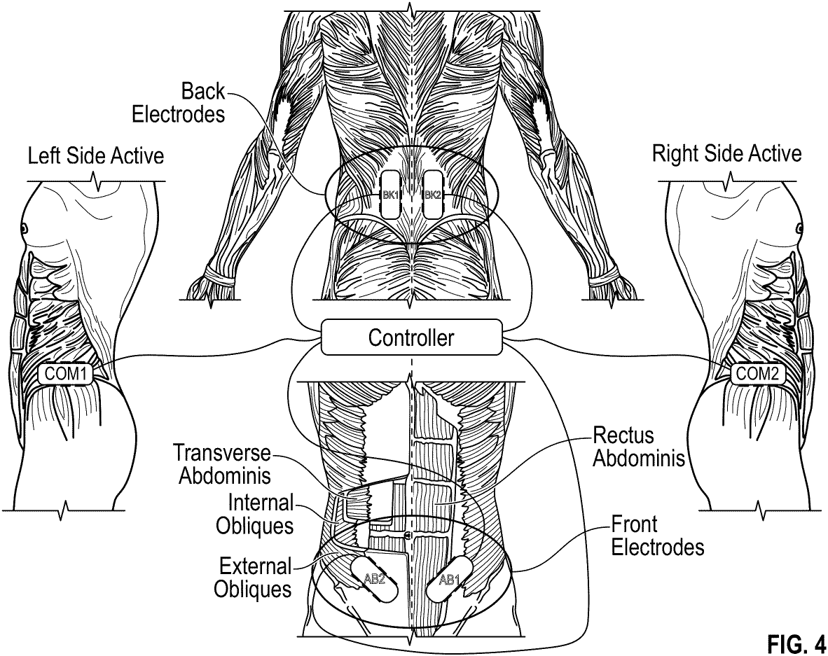

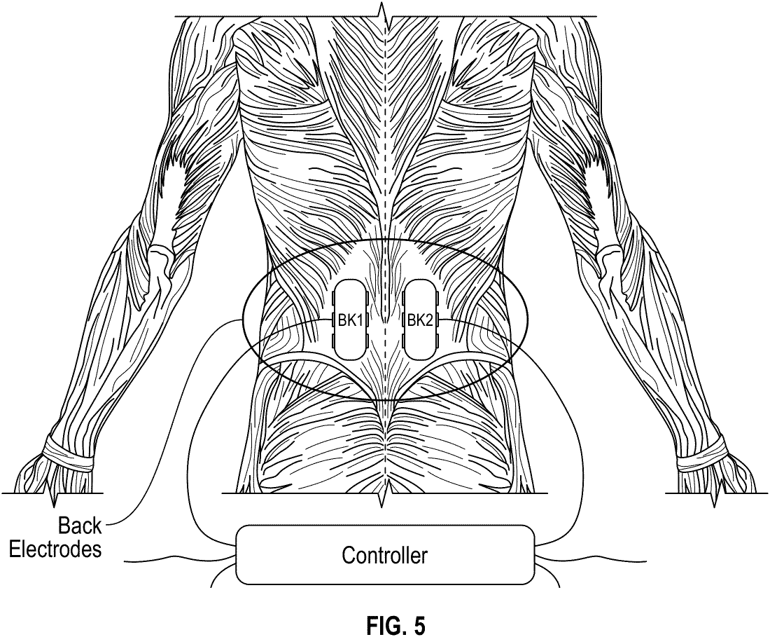

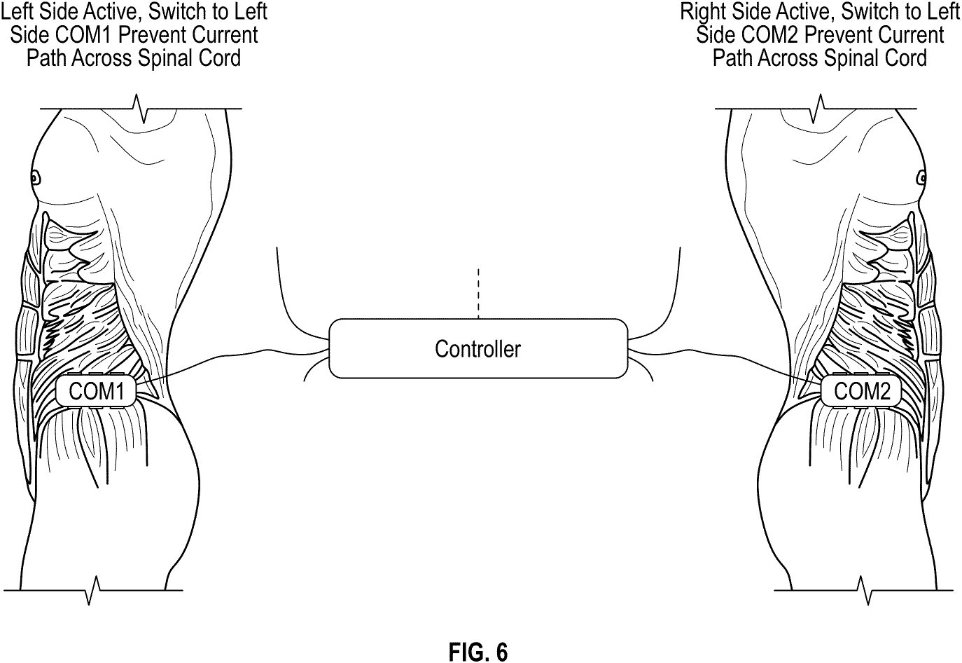

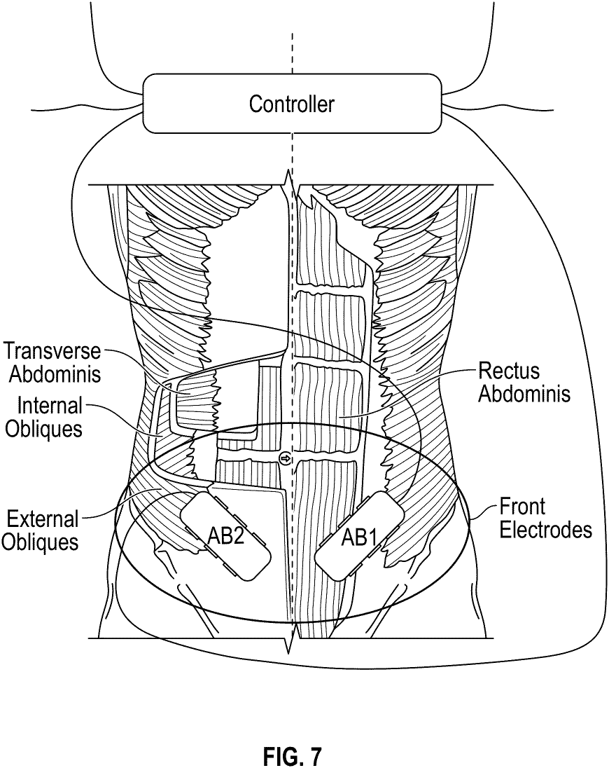

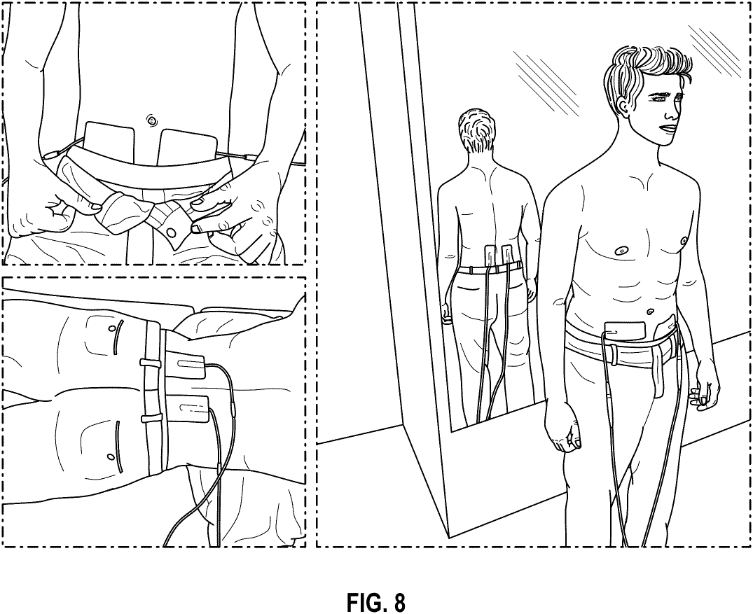

FIGS. 4-8 illustrates a method of electrode placement for delivery of the co-contraction program and/or CLPB treatment on a patient according to some embodiments. In some embodiments, a method step includes placing one or more electrodes on the abdominal area above the iliac crest and lower back at the L2 through L5 region. In some embodiments, activation of the electrodes in this area stimulates both TrA and LM muscles which are responsible for core strength and stability of the spinal column. In some embodiments, the system includes right and/or left activation of TrA and LM muscles to treat instability in the CLBP patients as previously described. In some embodiments, the system includes one or more program steps for activating the electrode in a pattern comprising an approximately 5 minute activation of the abdominal area, an approximately 5 minute activation of the back area, and/or an approximately 5 minute co-contraction of both the abdominal and back area.

In some embodiments, the system is configured to implement bilateral, independent, and/or concurrent muscle electrical stimulation of abdominal TrA and/or LM of the lower back. In some embodiments, the co-contraction program is configured to provide a unique NMES pulse train that includes approximately 5 minutes of a bilateral and independent TA muscles stimulation, approximately 5 minutes of a bilateral and independent LM muscles stimulation, and approximately 5 minutes of a bilateral and concurrent TrA/LM muscle stimulation. Although, on average, 5 minutes was found to obtain desired results, a range of 2 minutes to 8 minutes was found to be sufficient according to some embodiments. In some embodiments, the system is configured to repeat the pattern a programmed specified number of times and/or a specified time duration.

In some embodiments, the system is configured to execute a NMES activation protocol with a TENS activation protocol in a single therapeutic session. In some embodiments, the NMES activation protocol provides long-term benefits for core muscles (TA and LM) strengthening, restores muscle control, and reduces pain. In some embodiments, the TENS activation protocol provides the short-term and immediate benefits of pain reduction by blocking the pain signals traveling from the brain to the lower back area (i.e., the gate control theory of pain).

In some embodiments, the system includes a heating element. In some embodiments, the panel includes a heating element. In some embodiments, the garment includes a heating element. In some embodiments, the system is configured to execute a (dry) heat therapy program for lower back pain to provide an immediate comfort. In some embodiments, the heating element is configured to emit infrared rays, where Far Infrared Radiation (FIR) heat therapy is employed by the system, either alone or in combination with the co-contraction program and/or hybrid NMES/TENS protocol described herein.

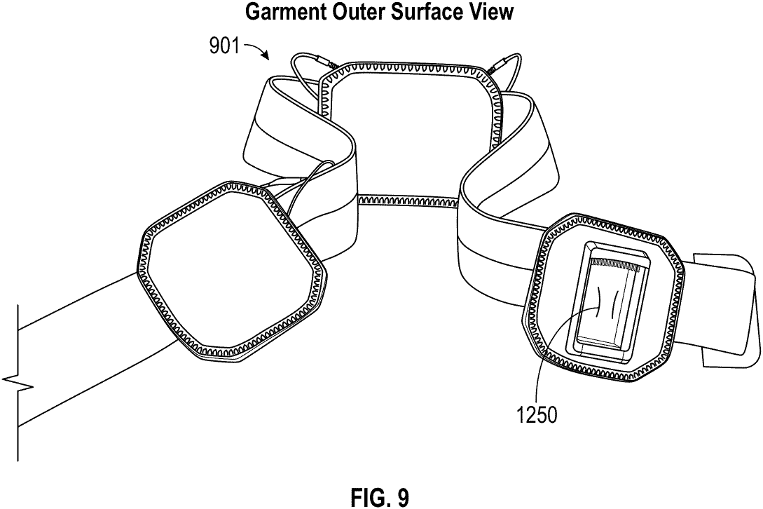

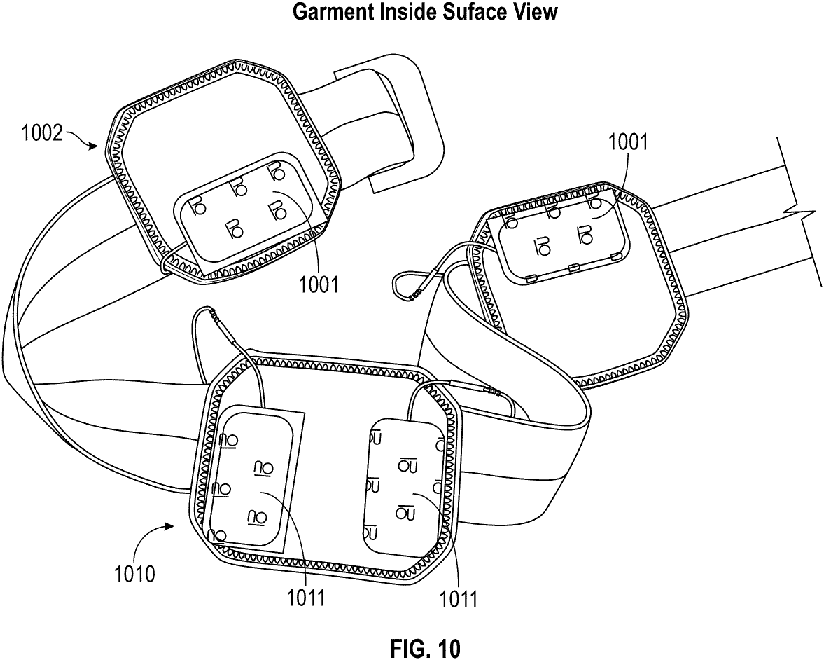

FIGS. 9 and 10 show a back and core wearable garment 901 that includes a unique wrap, band, or belt configured to ensure proper placement of one or more electrodes on the user to implement bilateral, independent, and/or concurrent muscle electrical stimulation of abdominal TrA and/or LM of the lower back. FIG. 9 depicts an outer surface view showing panel placement and/or controller dock 1250 location when the garment is viewed in third person as it is worn. FIG. 10 shows a garment inside surface view. In some embodiments, the inside surface view shows including transversus abdominus electrodes 1001 coupled to the abdominal panels 1002 adjustably positioned at outer extremities of the garment 901. In some embodiments, the inside surface view shows one or more back panels 1010 which include one or more rectus femoris electrodes 1011 configured to active LM muscles. In some embodiments, the one or more electrodes are secured to the garment 901 to position the one or more electrodes on a user as shown in FIGS. 4-8. In some embodiments, the garment 901 is configured to secure one or more back panels in position while allowing one or more abdominal panels to be adjusted.

In some embodiments, the wearable garment 901 includes one or more snap-type electrodes (e.g., up to 6) configured for placement over the TrA muscles of the abdomen and LM muscles of the lower back. In some embodiments, the wearable and/or panel includes a unique electrode template that includes 3 or more electrodes for the abdominal area configured to activate the right TrA, left TrA, and/or common; and/or a unique electrode template for the lower back configured to activate the right LM, left LM, and/or common. In some embodiments, the wearable garment includes magnetic auto-aligning electrodes snaps configured to hold one or more electrodes and/or one or more panels in position while enabling electrical energy transfer.

In some embodiments, the system includes a removable panel, also referred to as a smart panel herein. In some embodiments, the ability of a panel to be removed provides the benefit of allowing the garment 901 to be washed without damaging a panel and/or electrode. In some embodiments, each panel position is adjustable over the wearable (e.g., waste band). In some embodiments, the panel includes a foam construct substrate (e.g., Lycra® layer/open cell foam layer/loop layer) that includes electronics and/or printed flexible wires. In some embodiments, the electronics and/or printed flexible wires are embedded in the foam substrate. In some embodiments, the panel includes a unique electrode template that includes pre-positioned electrodes for NMES and/or TENS for abdominal and/or lower back stimulation.

In some embodiments, the panel and/or garment 901 includes conductive textile electrodes that are integrated in the panel assembly. In some embodiments, the garment 901 includes garment circuitry configured to link one or more panels to a single controller 1304. In some embodiments, the system includes a removable panel including electronics, electrodes, and sensors. In some embodiments, each of the one or more panel’s position is adjustable. In some embodiments, the system includes a wired or wireless snapped type pulse generator for electrical stimulation therapies and/or heat therapy. In some embodiments, the system includes one or more of an embedded force gauge, accelerometer, electromyogram (EMG), and pressure gauge in the wrap or Smart Panel to detect the activation forces of deep TrA muscles and LM muscle of the back. In some embodiments, the system includes a graphical user interface (GUI) configured to display activation levels of the TrA and LM muscles. In some embodiments, the GUI is visible by the user. In some embodiments, the controller is configured to calculate minimum, maximum, and/or required NMES stimulation intensities for the TrA and MF muscles based on the activation force of the TrA and LM muscles obtained from one or more sensors.

Some embodiments include systems and methods that provide co-contraction therapy to slow the progression of CLBP disease by predicting changes of the disease progression and spine and core health by measuring the muscle strength, and/or using patient database analytics and/or machine learning.

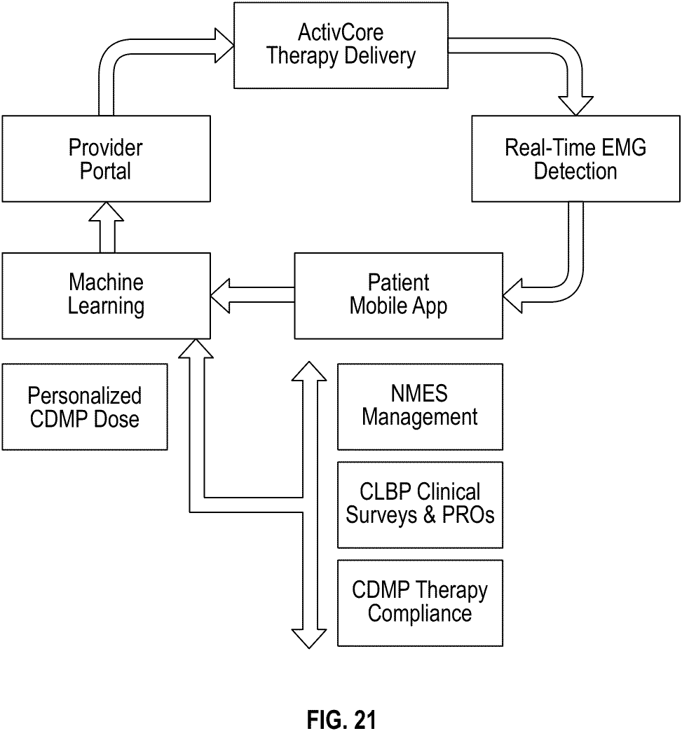

In some embodiments, the system is configured to execute an estimated NMES calculation configured to estimate a personalized dose of NMES therapy (intensity and/or duration). In some embodiments, the estimate calculation includes measuring a patient’s muscle strength, disease stage, and/or by using a patient database analytics and/or machine learning. FIG. 21 shows a non-limiting training workflow for machine learning for any NMES, TENS, and/or co-contraction programs described herein according to some embodiments.

In some embodiments, the system is configured to estimate correlations between or among patient demographics, NMES intensity, NMES duration, muscle strength, EMG, and/or CLBP pain level by application of the training workflow for machine learning algorithms (FIG. 21).

Some further embodiments include a biofeedback system for simultaneous detection of biofeedback signals of TrA and LM muscles’ activation by means of EMG, movement, or contraction detection using wearable wireless EMG sensors, pressure sensors, PZT sensors, force-sensitive sensor, strain gauge, etc.

In some embodiments, the system includes an integrated biofeedback platform that provides an interactive real-time monitoring and/or visualization of the muscle contractions and may include a portable control module configured to couple to the wearable garment for delivery of wireless EMS, control of EMS channels and intensities, collection, storage, and display of detected biofeedback signals (EMG, pressure, and movement). In some embodiments, the portable control module comprises an application (App) on a computer such as a mobile device.

In some embodiments, the system is configured to display a user’s compliance with a co-contraction program schedule and/or or is configured to correlate one or more sensed parameters (e.g., elevated heart rate, muscle contraction) to pain intensity for analytics. In some embodiments, the system is configured to calculate a quality of life score. In some embodiments, the system is configured to track, review, and share data with one or more providers. In some embodiments, the data may be analyzed in real time and feedback may be provided to the user based on the analysis. In some embodiments, the analysis may be used to alter behavior of the user and/or therapy. In some embodiments, the system is configured to alter the controller program instructions to implement a personalized therapy dose (e.g., intensity and/or duration) for NMES and/or TENS based on the collected health data and/or application of the machine learning algorithm’s recommendation.

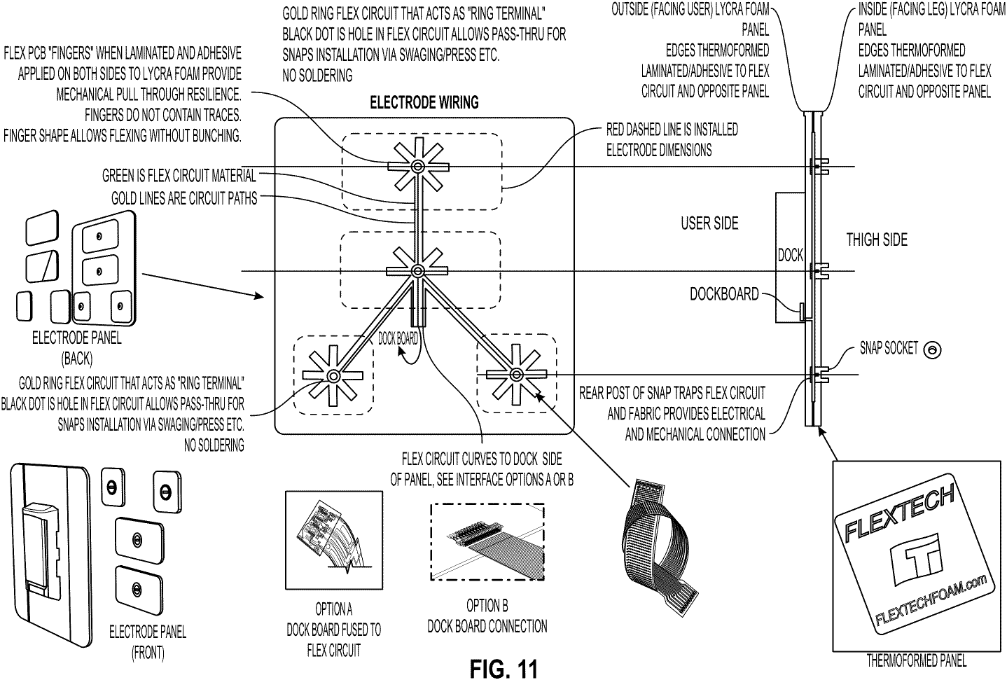

FIG. 11 illustrates a non-limiting system overview according to some embodiments. In some embodiments, the panel includes one or more pre-positioned removable electrodes. In some embodiments, the panel includes 3 rows of electrodes. In some embodiments, the first row comprises a single elongated electrode. In some embodiments, the second row comprises a single elongated electrode. In some embodiments, the third row comprises two electrodes, where the two electrodes are of equal size and shape. In some embodiments, each of the two electrodes include equal sides and/or radii.

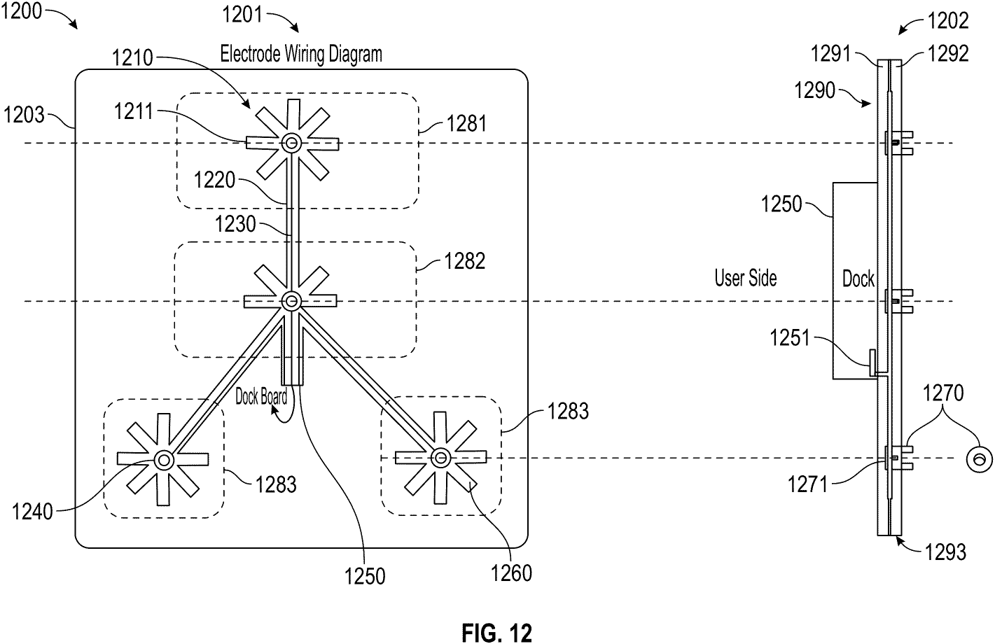

FIG. 12 shows an electrode wiring diagram 1200 according to some embodiments. In some embodiments, front view 1201 and side view 1202 depict a non-limiting configuration for an electrode panel 1203. In some embodiments, the panel 1203 comprises a flexible circuit 1210 comprising one or more flex PCB fingers 1211. In some embodiments, the flexible circuit 1210 comprises lamination and/or adhesive applied to one or both sides of the (Lycra® foam) panel 1290. In some embodiments, the flexible circuit 1210 is positioned between a user side (Lycra® foam) panel 1291 and a body side (Lycra® foam) panel 1292. In some embodiments, one or both panels 1291, 1292 comprise a thermoformed panel 1293. In some embodiments, the adhesive is configured to provide mechanical pull through resilience. In some embodiments, the mechanical pull is configured to return the panel to its original shape after being bent. In some embodiments, the fingers 1211 do not contain traces. In some embodiments, the shape of the fingers 1211 are configured to allow flexing without bunching.

In some embodiments, the front view 1201 depicts circuit material 1220 comprising circuit paths 1230 connecting electrodes 1281-1283 represented by dashed boxes. In some embodiments, the flexible circuit 1210 includes a ring terminal hole 1240 in the flexible circuit configured to enable connection to a snap socket 1270. In some embodiments, the snap socket 1270 is installed via swaging or pressing, for example, and/or does not require any soldering or adhesives. In some embodiments, a rear post 1271 of the snap socket 1270 traps the flexible circuit and fabric together and provides an electrical and/or mechanical connection. In some embodiments, the flexible circuit 1210 comprises a dock 1250 that curves to an interface with a dock board 1251 on the user side foam panel 1291.

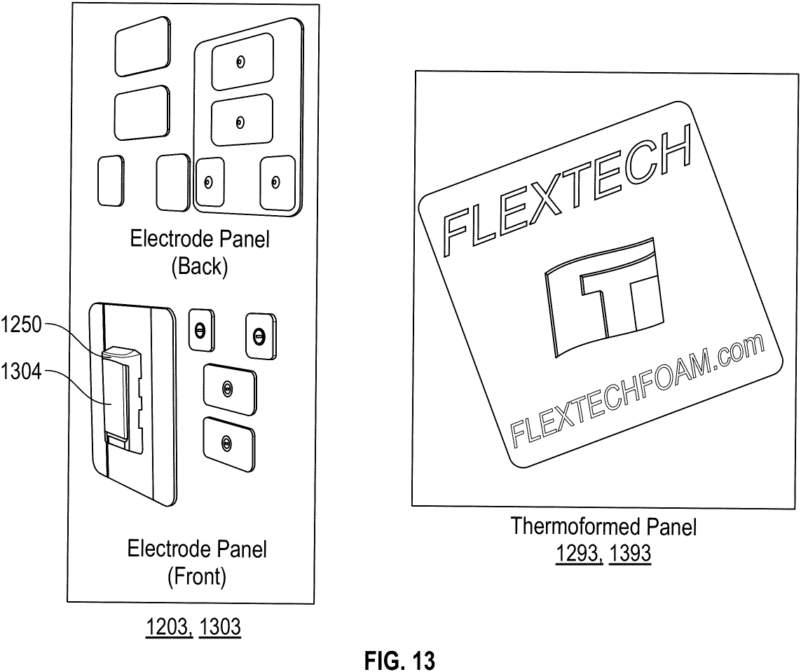

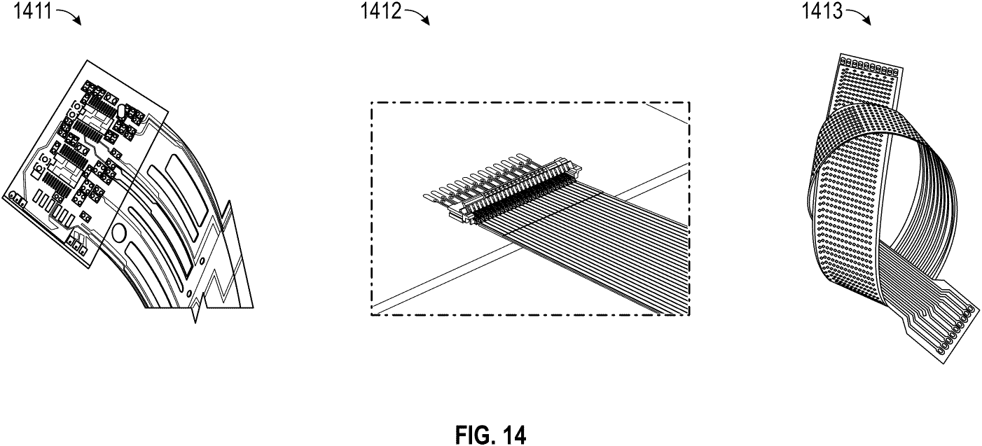

FIG. 13 depicts rendered views of the assembled electrical panel 1203, 1303 as well as the thermoformed panel 1293, 1393. In some embodiments, the dock 1250 is configured to couple to a controller 1304 and/or removably secure a controller 1304 in a fixed position. FIG. 14 shows options for connecting the docking board to the flexible circuit as well as a rendering of the flexible circuit material 1413. In some embodiments, a first option 1411 for connecting the dock board includes fusing the dock board to the flexible circuit. In some embodiments, a second option 1412 includes a detachable dock board connection. In some embodiments, benefits of the system include: no soldering, no sewing, no use of thermoplastic elastomers, low profile, flexes/contours on and around joints, provides structure/rigidity to electronics assembly supporting therapy application across multiple joints (on or around) through the modular system which includes garments and/or bands.

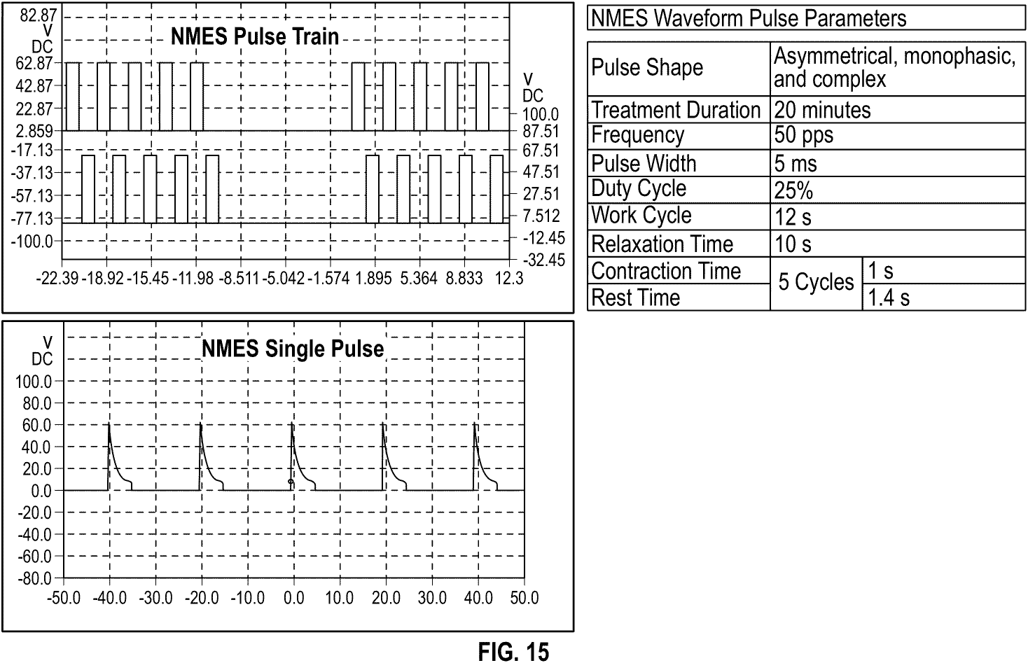

FIG. 15 shows NMES waveform parameters for the CLBP device described herein according to some embodiments. In some embodiments, the NMES waveform includes a unique, asymmetrical, complex, wide, and monophasic shaped pulse designed to provide improved therapeutic benefits. In some embodiments, the work cycle comprises the combination of five cycles of contraction and rest during a treatment cycle. In some embodiments, contraction time is the actual stimulation contraction period. In some embodiments, rest time is the period between contractions to wait to oscillate the stimulation between the two channels. In some embodiments, relaxation time is a period of no stimulation between the work cycles. In some embodiments, treatment duration includes a range of 10-30 minutes, where 20 minutes has been found to produce desired results. In some embodiments, a frequency for the work cycle includes a range of 25 to 75 pulses per second, where 50 pulses per second have been shown to produce desirable results. In some embodiments, a duty cycle includes a range of 15-35%, where around 25% has been shown to achieve particularly desirable results. In some embodiments, a work cycle, which includes a set of pulses, includes a range of 8 to 16 seconds, where around 12 seconds has been found to produce particularly desirable results. In some embodiments, a relaxation time, which includes a time between work cycles, includes a range of 7 to 13 seconds, where around 10 seconds has been shown to produce particularly desirable results. In some embodiments, a contraction time and/or rest time includes a range of 3-7 cycles, where around 5 cycles has been found to produce particularly desirable results.

In some embodiments, the system provides NMES concurrent stimulation. In some embodiments, the system provides a bilateral, independent, and concurrent muscle electrical stimulation of abdominal transverse abdominis and multifidus of lower back. In some embodiments, the system includes 5 minutes of a bilateral and independent TA muscles stimulation, 5 minutes of a bilateral and independent LM muscles stimulation, and 10 minutes of a bilateral and concurrent TrA/LM muscles stimulation. In some embodiments, concurrent stimulation is provided through pulse generator firmware updates by means of high frequency channel switching. In some embodiments, the design fully supports 4 channels (4 electrodes) of alternating stimulation.

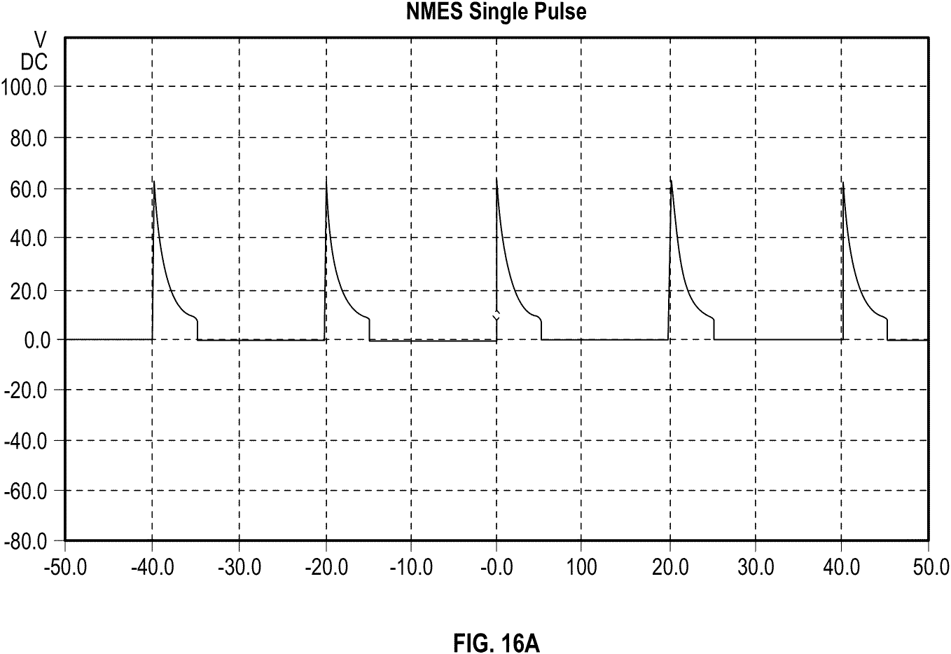

FIGS. 16A and 16B show NMES/TENS combination waveform parameters according to some embodiments. In some embodiments, the CLBP device pulse generator provides a combined NMES protocol with TENS protocol in a single therapeutic session. In some embodiments, the NMES protocol provides the long-term benefits of core muscle (TrA and LM) strengthening, restoring muscle controls, and pain reduction by stimulating the motor neurons of the lower back and abdomen. In some embodiments, the TENS protocol provides the short-term and immediate benefits of pain reduction by stimulating the sensory neurons of the lower back, blocking the pain signals traveling from the brain to the lower back area (gate control theory of pain).

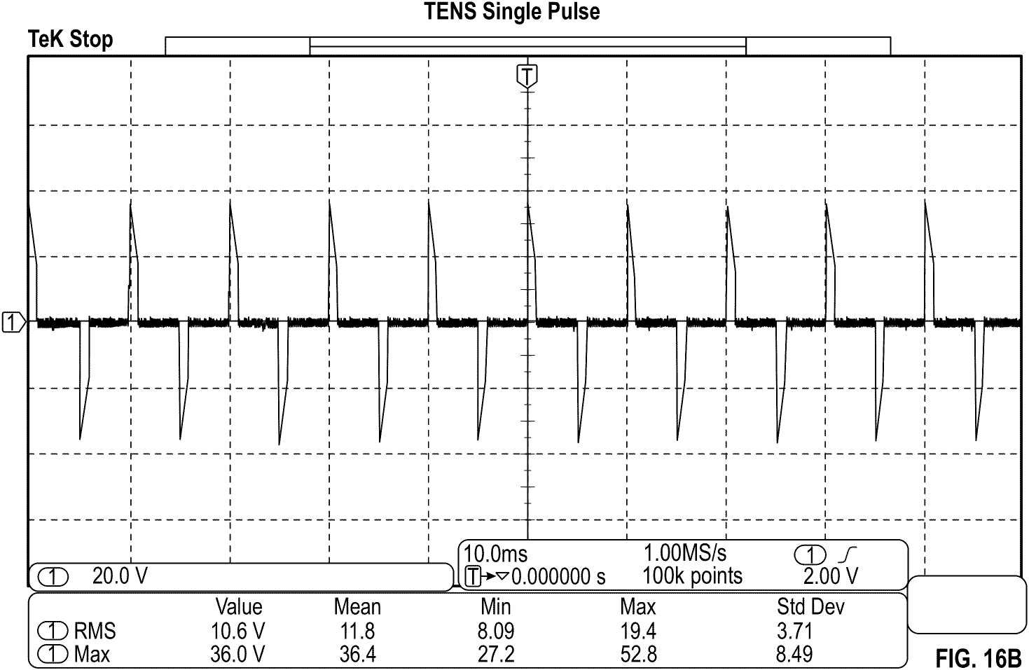

FIG. 17 depicts additional NMES/TENS combination waveform parameters according to some embodiments. In some embodiments, a single therapeutic session includes a total of 25 minutes combined NMES and TENS. First, in some embodiments, a 20-min NMES therapy session of TrA/LM is applied. Second, the pulse generator switches to TENS program providing 10 minutes of TENS therapy for lower back only according to some embodiments. In some embodiments, TENS therapy is applied during the “relaxation” cycles of NMES therapy (each relaxation time is 10 s), providing an interleaved therapy of NMES and TENS programs.

In some embodiments, the system includes a single printed control board (PCB) or other electronic assembly capable of producing NMES, heat output, and/or TENS output. In some embodiments, heat output is generated using a heating element or resistive heating element integrated in the back portion of the lower back band, which is configured to apply the electrodes and/or heating elements on the L4/L5 area of the lower back. In some embodiments, the heating element(s) are positioned away from the LM electrodes. In some embodiments, the heating element(s) at least partially overlap with the LM electrodes.

In some embodiments, the system includes a controller with program instructions that configure the controller to implement a single therapeutic session that includes a total of 30 minutes combined NMES or TENS and heat therapy, which may be programmed by the user according to some embodiments. In some embodiments, the controller is configured to apply a 20-min NMES and/or TENS therapy of TrA/LM region as a first step. In some embodiments, the controller is configured to shut off the NMES/TENS pulse generator, and the same printed control board assembly (PCBA) generates heat using heat elements and/or heat sensors providing 10 minutes of thermotherapy for the lower back pain as a second step. In some embodiments, the system includes one or more thermistor sensors to gauge and apply temperature safely. In some embodiments, the system is configured to apply heat to the lower back area above the average skin temperature of 37° C., which includes a range of 32 to 42 degrees Celsius according to some embodiments.

Some embodiments of the system were tested on three subjects with the following characteristics: body mass indexes (BMIs) as high as 31.5, male and female, CLBP and healthy subjects. In some embodiments, activation of the TrA and LM muscles were confirmed with ultrasound. In some embodiments, stimulation of the TrA and LM muscles was feasible for high BMI subjects (2 of the 3 had a BMI of 29 and 31.5). In some embodiments, stimulation sensation of the abdominal and lower back areas was comfortable for all three subjects. In some embodiments, increase of TrA and LM muscle thickness was observed for all three subjects during NMES application. FIG. 18 depicts a change in TrA thickness from rest to stimulation according to some embodiments. FIG. 19 shows a change in LM thickness from rest to stimulation according to some embodiments.

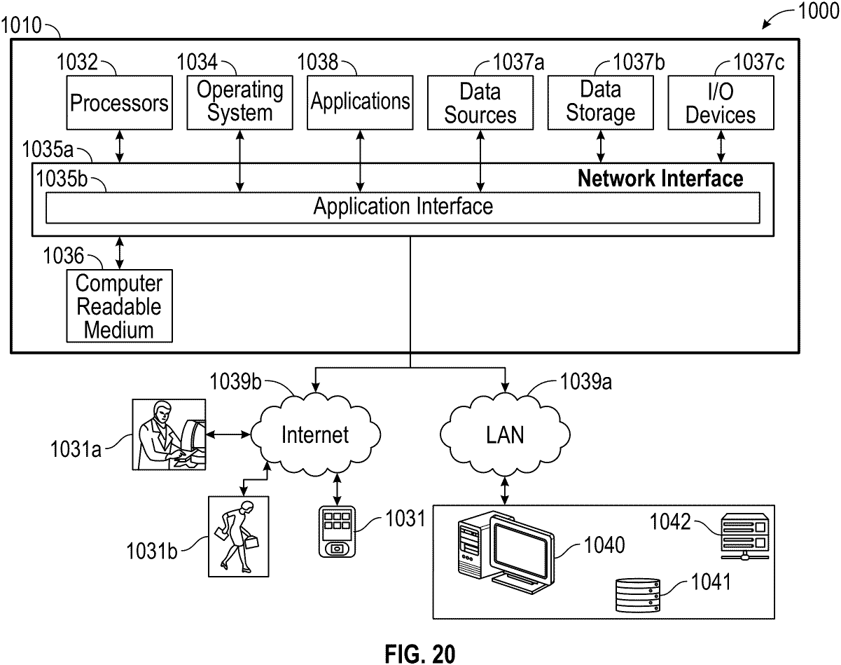

FIG. 20 illustrates a computer system 1010 enabling or comprising the systems and methods in accordance with some embodiments of the system. In some embodiments, the computer system 1010 can operate and/or process computer-executable code of one or more software modules of the aforementioned system and method. Further, in some embodiments, the computer system 1010 can operate and/or display information within one or more graphical user interfaces (e.g., HMIs) integrated with or coupled to the system.

In some embodiments, the computer system 1010 can comprise at least one processor 1032. In some embodiments, the at least one processor 1032 can reside in, or be coupled to, one or more conventional server platforms (not shown). In some embodiments, the computer system 1010 can include a network interface 1035a and an application interface 1035b coupled to the at least one processor 1032 capable of processing at least one operating system 1034. Further, in some embodiments, the interfaces 1035a, 1035b coupled to at least one processor 1032 can be configured to process one or more of the software modules (e.g., such as enterprise applications 1038). In some embodiments, the software application modules 1038 can include server-based software and can operate to host at least one user account and/or at least one client account and operate to transfer data between one or more of these accounts using the at least one processor 1032.

With the above embodiments in mind, it is understood that the system can employ various computer-implemented operations involving data stored in computer systems. Moreover, the above-described databases and models described throughout this disclosure can store analytical models and other data on computer-readable storage media within the computer system 1010 and on computer-readable storage media coupled to the computer system 1010 according to various embodiments. In addition, in some embodiments, the above-described applications of the system can be stored on computer-readable storage media within the computer system 1010 and on computer-readable storage media coupled to the computer system 1010. In some embodiments, these operations are those requiring physical manipulation of physical quantities. Usually, though not necessarily, in some embodiments these quantities take the form of one or more of electrical, electromagnetic, magnetic, optical, or magneto-optical signals capable of being stored, transferred, combined, compared and otherwise manipulated. In some embodiments, the computer system 1010 can comprise at least one computer readable medium 1036 coupled to at least one of at least one data source 1037a, at least one data storage 1037b, and/or at least one input/output 1037c. In some embodiments, the computer system 1010 can be embodied as computer readable code on a computer readable medium 1036. In some embodiments, the computer readable medium 1036 can be any data storage that can store data, which can thereafter be read by a computer (such as computer 1040). In some embodiments, the computer readable medium 1036 can be any physical or material medium that can be used to tangibly store the desired information, data or instructions and which can be accessed by a computer 1040 or processor 1032. In some embodiments, the computer readable medium 1036 can include hard drives, network attached storage (NAS), read-only memory, random-access memory, FLASH-based memory, CD-ROMs, CD-Rs, CD-RWs, DVDs, magnetic tapes, other optical and non-optical data storage. In some embodiments, various other forms of computer-readable media 1036 can transmit or carry instructions to a remote computer 1040 and/or at least one user 1031, including a router, private or public network, or other transmission or channel, both wired and wireless. In some embodiments, the software application modules 1038 can be configured to send and receive data from a database (e.g., from a computer readable medium 1036 including data sources 1037a and data storage 1037b that can comprise a database), and data can be received by the software application modules 1038 from at least one other source. In some embodiments, at least one of the software application modules 1038 can be configured within the computer system 1010 to output data to at least one user 1031 via at least one graphical user interface rendered on at least one digital display.

In some embodiments, the computer readable medium 1036 can be distributed over a conventional computer network via the network interface 1035a where the system embodied by the computer readable code can be stored and executed in a distributed fashion. For example, in some embodiments, one or more components of the computer system 1010 can be coupled to send and/or receive data through a local area network (“LAN”) 1039a and/or an internet coupled network 1039b (e.g., such as a wireless internet). In some embodiments, the networks 1039a, 1039b can include wide area networks (“WAN”), direct connections (e.g., through a universal serial bus port), or other forms of computer-readable media 1036, or any combination thereof.

In some embodiments, components of the networks 1039a, 1039b can include any number of personal computers 1040 which include for example desktop computers, and/or laptop computers, or any fixed, generally non-mobile internet appliances coupled through the LAN 1039a. For example, some embodiments include one or more of personal computers 1040, databases 1041, and/or servers 1042 coupled through the LAN 1039a that can be configured for any type of user including an administrator. Some embodiments can include one or more personal computers 1040 coupled through network 1039b. In some embodiments, one or more components of the computer system 1010 can be coupled to send or receive data through an internet network (e.g., such as network 1039b). For example, some embodiments include at least one user 1031a, 1031b, is coupled wirelessly and accessing one or more software modules of the system including at least one enterprise application 1038 via an input and output (“I/O”) 1037c. In some embodiments, the computer system 1010 can enable at least one user 1031a, 1031b, to be coupled to access enterprise applications 1038 via an I/O 1037c through LAN 1039a. In some embodiments, the user 1031 can comprise a user 1031a coupled to the computer system 1010 using a desktop computer, and/or laptop computers, or any fixed, generally non-mobile internet appliances coupled through the internet 1039b. In some embodiments, the user can comprise a mobile user 1031b coupled to the computer system 1010. In some embodiments, the user 1031b can connect using any mobile computer 1031c to wireless coupled to the computer system 1010, including, but not limited to, one or more personal digital assistants, at least one cellular phone, at least one mobile phone, at least one smart phone, at least one pager, at least one digital tablets, and/or at least one fixed or mobile internet appliances.

The subject matter described herein are directed to technological improvements to the field of lower back pain mitigation through new arrangements and applications of NMES. The disclosure describes the specifics of how a machine including one or more computers comprising one or more processors and one or more non-transitory computer readable media implement the system and its improvements over the prior art. The instructions executed by the machine cannot be performed in the human mind or derived by a human using a pen and paper but require the machine to convert process input data to useful output data. Moreover, the claims presented herein do not attempt to tie-up a judicial exception with known conventional steps implemented by a general-purpose computer; nor do they attempt to tie-up a judicial exception by simply linking it to a technological field. Indeed, the systems and methods described herein were unknown and/or not present in the public domain at the time of filing, and they provide technologic improvements and advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Furthermore, acting as Applicant’s own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

As used herein, “can” or “may” or derivations thereof (e.g., the system display can show X) are used for descriptive purposes only and is understood to be synonymous and/or interchangeable with “configured to” (e.g., the computer is configured to execute instructions X) when defining the metes and bounds of the system.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with “operatively connected” when used in conjunction with physical structures.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures, have like reference numerals. The figures, which are not necessarily to scale, depict some embodiments and are not intended to limit the scope of embodiments of the system.

Any of the operations described herein that form part of the system are useful machine operations. The system also relates to a device or an apparatus for performing these operations. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network, the data can be processed by other computers on the network, e.g., a cloud of computing resources.

The embodiments of the system can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods which can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes, without limitation, volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the system has been described above in connection with particular embodiments and examples, the system is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the system are set forth in the following claims.

We claim:

1. A system for alleviating chronic lower back pain comprising:
a wearable garment, and
a plurality of electrodes;
wherein the plurality of electrodes are positioned on the wearable garment such that a neuromuscular electrical stimulation (NMES) pulse to each of the plurality of electrodes will simultaneously contract transversus abdominis (TrA) muscles and lumbar multifidus (LM) muscles.

2. The system of claim 1,
wherein the wearable garment is configured to enable one or more of the plurality of electrodes to be repositioned.

3. The system of claim 1, further comprising:
a panel;
wherein the panel comprises at least one of the plurality of electrodes; and
wherein the panel is configured to electrically couple to at least one of the plurality of electrodes.

4. The system of claim 3,
wherein the panel is configured to add rigidity to at least one of the plurality of electrodes.

5. The system of claim 3,
wherein the wearable garment is configured to secure the panel in position such that at least one of the plurality of electrodes contracts at least one of the TrA muscles and the LM muscles when an NMES pulse is applied to at least one of the plurality of electrodes.

6. The system of claim 1, further comprising:
a plurality of panels;
wherein each of the plurality of panels comprise electrical contacts configured to physically couple to at least one of the plurality of electrodes;
wherein each of the plurality of panels are configured to electrically couple to at least one of the plurality of electrodes; and
wherein the wearable garment is configured to secure each of the plurality of panels in a position such that an NMES pulse contracts the TrA muscles and/or the LM muscles.

7. A system for alleviating chronic lower back pain comprising:
an electrode panel;
wherein the electrode panel comprises electrical connections and/or mechanical connections for two or more electrodes.

8. The system of claim 7,
wherein the mechanical connections are configured to position the two or more electrodes such that a neuromuscular electrical stimulation (NMES) pulse to each of the two or more electrodes will contract transversus abdominis (TrA) muscles.

9. The system of claim 8,
wherein the mechanical connections are configured to position the two or more electrodes such that a neuromuscular electrical stimulation (NMES) pulse to each of the two or more electrodes will contract lumbar multifidus (LM) muscles.

10. The system of claim 9, further comprising:
a controller is configured to:
execute, by one or more processors, a TrA pulse;
stop, by the one or more processors, the TrA pulse;
execute, by the one or more processors, an LM pulse;
stop, by the one or more processors, the LM pulse; and
execute, by the one or more processors, the TrA pulse and the LM pulse, simultaneously.

11. The system of claim 7,
wherein the electrode panel comprises rigidity properties and flexibility properties;
wherein the rigidity properties enable the electrode panel to maintain a substantially same shape under an influence of gravity when rotated to various positions; and
wherein the flexibility properties enable the electrode panel to bend an amount sufficient to ensure each of the two or more electrodes contact skin to contract transversus abdominis (TrA) muscles and/or lumbar multifidus (LM) muscles when the two or more electrodes are connected to the electrode panel.

12. The system of claim 7,
wherein the electrode panel comprises an electrical circuit configured to deliver electrical pulses to each of the two or more electrodes;
wherein the electrode panel comprises a dock configured to enable a controller to be connected to a docking board.

13. The system of claim 12, further comprising:
a controller, and
a garment;
wherein the controller is configured to electrically couple to the electrode panel; and
wherein the garment comprises one or more mechanical couplings configured to removably secure the controller and/or the electrode panel in a position.

14. A system for alleviating chronic lower back pain comprising:
a plurality of electrodes, and
a controller;
wherein the controller is configured to execute a neuromuscular electrical stimulation (NMES) program configured to generate one or more NMES pulses to each of the plurality of electrodes;
wherein the one or more NMES pulses comprise a transversus abdominis (TrA) pulse configured to contract TrA muscles; and
wherein the one or more NMES pulses comprise a lumbar multifidus (LM) pulse configured to contract LM muscles.

15. The system of claim 14,
wherein the controller is configured to:
execute, by one or more processors, the TrA pulse;
stop, by the one or more processors, the TrA pulse;
execute, by the one or more processors, the LM pulse; and
stop, by the one or more processors, the LM pulse.

16. The system of claim 14,
wherein the controller is configured to:
execute, by one or more processors, the TrA pulse;
stop, by the one or more processors, the TrA pulse;
execute, by the one or more processors, the LM pulse;
stop, by the one or more processors, the LM pulse; and
execute, by the one or more processors, the TrA pulse and the LM pulse simultaneously.

17. The system of claim 14,
wherein the controller is configured to execute transcutaneous electrical stimulation (TENS) and NMES programs.

18. The system of claim 14,
wherein the controller is configured to execute transcutaneous electrical stimulation (TENS) and NMES programs to a same electrode simultaneously.

19. The system of claim 14, further comprising:
a garment, and
one or more panels;
wherein the controller is configured to electrically couple to at least one of the one or more panels;
wherein the garment comprises one or more mechanical couplings configured to removably secure the one or more panels in one or more positions; and
wherein the one or more positions are configured to deliver the TrA pulse and/or the LM pulse.

20. The system of claim 19,
wherein the one or more panels comprise an electrical circuit configured to deliver electrical pulses to each of the plurality of electrodes; and
wherein the one or more panels comprises a dock configured to enable the controller to be connected to a docking board.
